(19) United States
(12) Patent Application Publication
(10) Pub. No.: US 2008/0039878 A1
(43) Pub. Date: Feb. 14, 2008

(54) Systems and methods for restoring function of diseased bowel

(76) Inventors: Michael S. Williams, Santa Rosa, CA (US); Daniel W. Fifer, Windsor, CA (US); William L. Athas, Chapel Hill, NC (US); Richard S. Stack, Chapel Hill, NC (US); Aurora Pryor, Durham, NC (US)

Correspondence Address: STALLMAN & POLLOCK LLP;Attn: Kathleen A. Frost, Suite 2200, 353 Sacramento Street, San Francisco, CA, 94111, US

(21) Appl. No.: 11/825,464

(22) Filed: Jul. 6, 2007

Related U.S. Application Data
(60) Provisional application No. 60/818,725, filed on Jul. 6, 2006.

(51) Int. Cl. A61F 2/04

(52) U.S. Cl. 606/153; 623/23.64

(57) ABSTRACT

In a first embodiment of a system for treating diseased bowel, a pair of incisions are formed on opposite ends of a diseased section of bowel. A tubular bypass implant is positioned in the bowel such that its ends are anchored within the bowel and such that an intermediate section of the implant is positioned external to the bowel such that bowel contents flow through the implant and thus around the diseased bowel section. In a second embodiment, the diseased section of bowel is removed and a system is implanted for joining limbs of the resected bowel together to form an anastomosis. In the preferred anastomosis system, the limbs of the resected bowel are positioned between a tubular sleeve extending through the bowel and a tubular cuff positioned around the bowel.

### RELATED APPLICATIONS

This application claims the benefit of U.S. Provisional Application No. 60/818,765, filed Jul. 6, 2006.

### TECHNICAL FIELD OF THE INVENTION

The present invention relates to systems and methods for restoring function to a bowel that is obstructed by a tumor or other disease condition.

### BACKGROUND

Disease states causing bowel obstruction include colon cancer, metastatic disease that has spread to the bowel, and other forms of cancer, including pancreatic cancer that invades the walls of the bowel. Patients suffering from bowel obstruction often must undergo a surgical procedure to relieve the obstruction in order to restore bowel function. One such procedure involves a bowel resection in which a tumor or a diseased section of the bowel is removed, and the remaining portions of the bowel are joined together to form an anastomosis. If the tumor or diseased section cannot be removed, one or more bypasses around the affected area are created by resecting sections of the bowel and performing an anastomosis by suturing the remaining limbs of the resected bowel.

This application describes a simplified system for joining limbs of the resected bowel together to form an anastomosis. This application further describes a bypass implant that may be used as an alternative to the conventional bypass procedure requiring resection and reassembly of the intestine.

### BRIEF DESCRIPTION OF THE DRAWINGS

FIG. 1 is a schematic illustration of a human digestive system, including the stomach, small intestine, and colon.

FIG. 2 is a plan view of an embodiment of an intestinal bypass device.

FIG. 3A is a schematic view of a portion of an intestine, illustrating placement of the bypass device of FIG. 2.

FIG. 3B is a cross-section view taking along the plane designated 3B-3B in FIG. 3A, showing the position of an end section of the bypass device of FIG. 2 within the intestine.

FIGS. 4A-4C are a sequence of drawings illustrating implantation of the bypass device of FIG. 2.

FIGS. 5A-5D are a sequence of drawings illustrating use of an anastomosis system, in which:

FIG. 5A is a side elevation view of a portion of an intestine prior to resection of a diseased section of the intestine;

5B is a side elevation view of the portion of the intestine shown in FIG. 5A after resection, showing the step of positioning the outer sleeve of the anastomosis system following resection of the diseased portion;

FIG. 5C is a cross-sectional side elevation view of the portion of the intestine, showing deployment of the inner sleeve of the anastomosis system; and

FIG. 5D is similar to FIG. 5C and shows the inner and outer sleeves following placement.

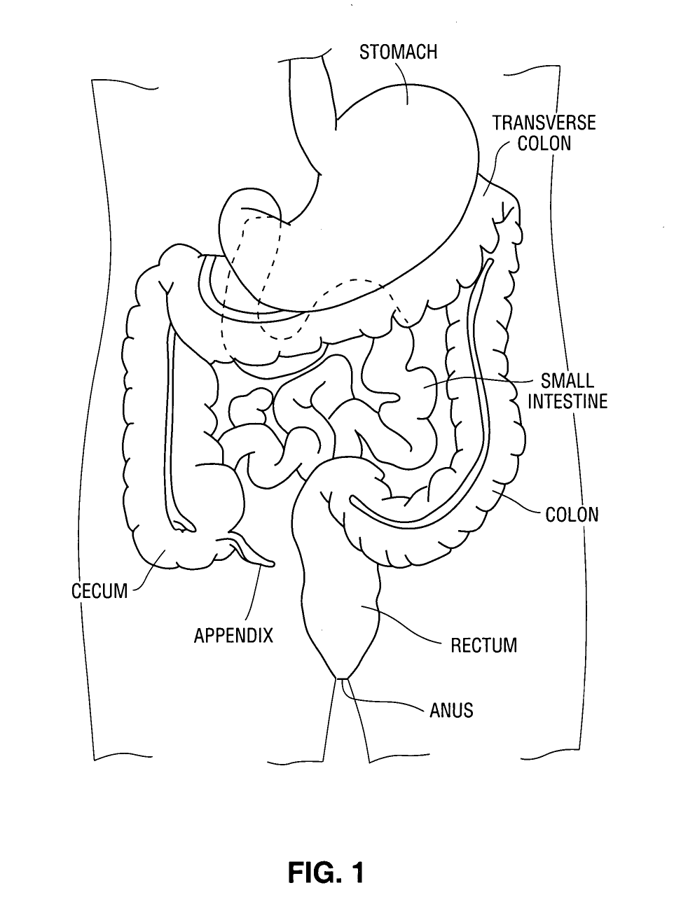

### DETAILED DESCRIPTION

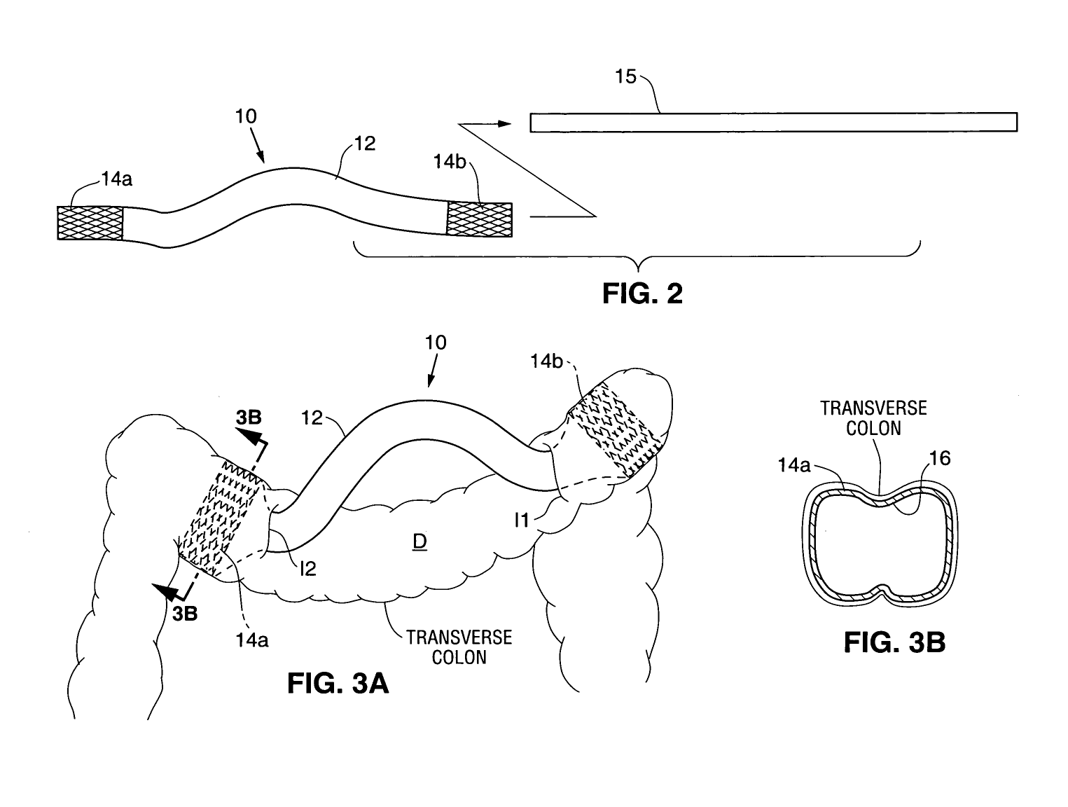

FIG. 2 shows a first embodiment of a bypass device 10 for use in restoring function to a diseased bowel. Bypass device 10 includes an elongate flexible tube 12 formed of a length of tubular material including but not limited to ePTFE graft material, other polymeric tubing or fluid-tight woven polymeric material (e.g. of the type used for vascular grafts). Expandable tubular anchors 14a, 14b are positioned on each end of the flexible tube such that a continuous lumen is formed through the anchors 14a, 14b and tube 12. The anchors may be attached to the tube 12, or they may be attachable to the tube during implantation. Preferred anchors are formed of a material that, when positioned within a body lumen, are capable of self-expanding or being expanded into sealing contact with the walls of the lumen. Suitable materials include but are not limited to shape memory (e.g. nickel titanium alloy, nitinol or shape memory polymer) elements or stainless steel, Eligoy, or MP35N wires or structures. Each anchor may include a polymeric material on its interior and/or exterior surface, and/or the structure of the anchor (e.g. mesh, braid etc.) may be impregnated with polymeric material. In a preferred embodiment, the flexible tube 12 and the anchors 14a, 14b are radially collapsible to a reduced diameter to facilitate deployment. The device 10 may be positioned in a removable sheath 15 to maintain the reduced diameter position during deployment.

The sizes of the tube 12 and anchors 14a, 14b will depend on the application, but one exemplary device may have a diameter of approximately 30 mm and a length of approximately 5 inches.

FIG. 3A shows the bypass device 10 after it has been positioned to divert bowel contents around a diseased region D of the transverse colon. The anchors 14a, 14b are disposed within the colon and make sealing contact with the walls of the colon as shown in FIG. 3B, thus insuring that material moving through the bowel passes into the lumen 16 of the device 10.

According to one method of implanting the bypass device 10, incisions II, I2 are formed in the colon using an instrument 17 (e.g. mechanical or electrosurgical blade, needle, etc.) introduced into the intestinal system via the rectum and advanced through the colon. Natural orifice access systems that may be employed to access the bowel transorally or transrectally, visualize the bowel, and/or form an incision in the bowel wall are shown and described in U.S. application Ser. No. 11/528,009, filed Sep. 27, 2006, which is also incorporated herein by reference.

The bypass device is retained in a radially-compressed configuration for advancement through the intestinal system. For example, the device 10 may be compressed and inserted into elongate sheath 15. After the incisions are formed, the bypass device 10 is advanced to the implant location, preferably under endoscopic visualization, using a catheter 19 or other endoscopic device passed through the rectum. In a preferred deployment method, the bypass device 10 (with the elongate sheath 15 maintaining its compressed state) is passed through a delivery lumen in a catheter 19 having features allowing the catheter to be advanced or steered through the colon. Features of this type are found on colonoscopy devices used for diagnosis and other procedures such as polypectomy. In an alternate deployment method, the bypass device 10 and sheath may be axially advanced over a catheter or other endoscopic device advanced or to be advanced to the implantation site.

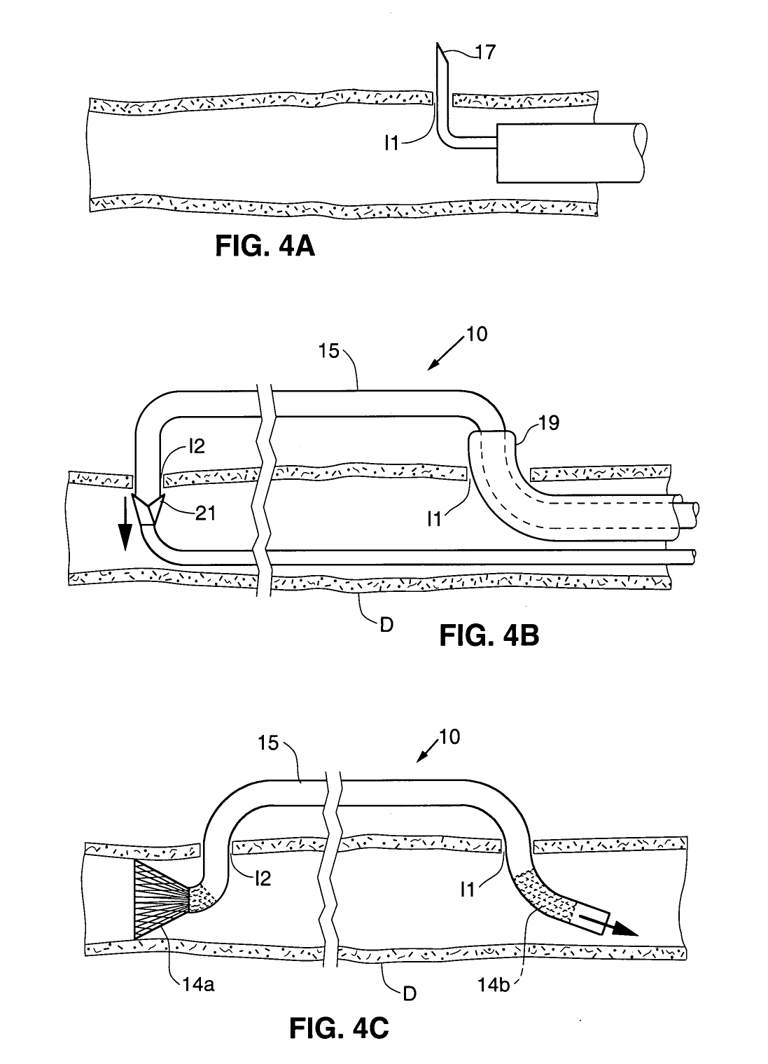

The leading end of the bypass device 10, preferably still packaged within compression sheath 15, is advanced out of the colon via one of the incisions II, and is then fed back into the colon through the other incision I2 as shown in FIG. 4B. Graspers or other instruments may be passed through the diseased portion of the bowel D and used to retrieve the leading end of the device from outside the incision I2 and withdraw it further into the colon, or to help advance the leading end further into the colon after it has been steered through the incision I2 using the catheter. Alternatively, instruments may be passed through a port or incision formed in the abdominal wall or through the umbilicus and used to assist in passing the leading end between the incisions I1, I2. Systems for supporting instruments in single port access procedures are shown and described in U.S. application Ser. No. 11/804,063, filed May 17, 2007, which is fully incorporated herein by reference.

Once the leading end of the device is in the desired location of the bowel, the sheath is partially withdrawn to expose the anchor 14a as shown in FIG. 4C. The anchor preferably expands into contact with the wall of the bowel as shown in FIG. 3B. Once it is confirmed that the trailing end of the device 10 is properly positioned, the sheath 15 is withdrawn further to expose the anchor 14b, allowing it to seal against the interior wall of the bowel. Removal of the sheath 15 also allows the flexible tube 12 to expand to its full diameter into the position shown in FIG. 3A.

In a slightly modified embodiment, the system may include a pair of separately-removeable sheaths, one of which confines the anchor 14a and the other of which confines the anchor 14b. According to this modified embodiment, the sheath covering anchor 14a would be withdrawn following positioning of the anchor 14a, and the sheath covering anchor 14b would be removed after anchor 14b is positioned. Either of the sheaths might be positioned to cover the flexible tube 12.

Adhesives may be optionally used to aid in fixing the anchors within the lumen of the bowel. For example, once the anchors are positioned, an adhesive may be applied through the anchor into contact with the inner surface of the bowel. Alternatively, the anchors may include adhesives activated by light, chemical interaction, or other means once the anchors are positioned within the bowel.

The device 10 and sheath 15 may be provided as a system together with additional instruments, which may include but are not limited to cutting element 17, catheter 19, grasper 21 and/or other instruments useful for implanting the device 10. The system may additionally be provided with instructions for use instructing the user to implant the device using methods disclosed herein and/or their equivalents.

In an alternate procedure, the bypass device 10 may be introduced laparoscopically, or using a single port formed in the abdominal wall or through the umbilicus. For example, the instruments for accessing and visualizing the bowel for forming the incision and deploying the implant 10 may be passed through the single port in the abdominal wall.

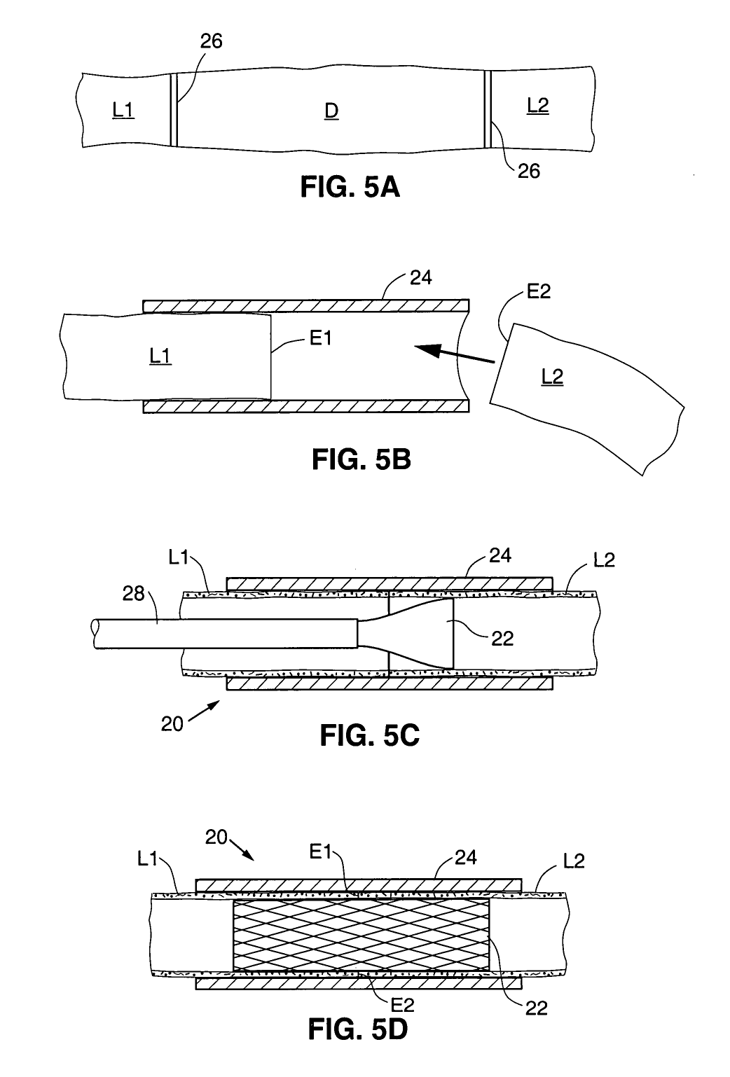

A second system 20 for restoring function to a diseased bowel is shown in use in FIG. 5D. The system 20 includes a tubular implant 22 positionable within a pair of resected limbs L1, L2 of bowel having resected edges E1, E1, and an outer cuff 24 positionable around the limbs L1, L2. Tubular implant 22 is preferably formed of a material that, when positioned within a body lumen, allows the implant to self-expand or to be actively expanded (i.e. using a balloon or other device expanded within the lumen of the implant 22) into contact with the walls of the bowel lumen as shown in FIG. 5D. Materials useful for the implant 22 include but are not limited to shape memory (e.g. nickel titanium alloy, nitinol or shape memory polymer) elements or stainless steel, Eligoy, or MP35N wires or structures including meshes or braids. The implant may be coated with a polymeric material on its interior and/or exterior surface, or it may be impregnated with polymer.

Cuff 24 may be formed of a polymeric material or other suitable biocompatible materials. Examples include but are not limited woven or porous polymers. The cuff 24 and implant 22 are proportioned such that they will engage the limbs L1, L2 between them, holding the resected edges E1, E2 in contact with one another, thus helping the edges to heal together. The cuff 24 and/or implant 22 may have a material or substance on its surface (e.g., the inner surface of the cuff or the outer surface of the implant) to facilitate healing.

Methods for performing an anastomosis using the system 20 may be carried out using natural orifice access (e.g. transoral or transrectal) and/or laparoscopic access. In one method of forming an anastomosis using the system 20, a single incision or port is formed in the abdominal wall or umbilicus. Instruments for accessing and visualizing the bowel for resection and deployment of the cuff are passed through the single port. Single port access systems of the type shown and described in U.S. Application Ser. No. 11/804,063, filed May 17, 2007 and incorporated herein by reference may be used for this purpose. According to this method, diseased section D of the bowel is closed off from the adjacent limbs L1, L2 using clips, sutures or other devices 26. Diseased section D is then resected from the bowel using a cutting instrument such as a mechanical or electrosurgical blade or other cutting element. Cutting instruments suitable for this purpose include but are not limited to those currently in use for bowel resection procedures. Next, the limb sections L1, L2 are inserted into the cuff 24 as shown in FIG. 5B and advanced longitudinally to position edges E1, E2 in contact with one another.

Once the edges are approximated, implant 22 is deployed within the limbs. This step may be carried out by compressing the implant into a catheter 28 and then passing the catheter into the bowel via the rectum (or, if the diseased section is in the small intestine, using transoral access through the mouth and stomach into the intestine). Natural orifice access systems that may be employed to access the bowel transorally or transrectally, visualize the bowel, and/or form an incision in the bowel wall are shown and described in U.S. application Ser. No. 11/528,009, filed Sep. 27, 2006, which is also incorporated herein by reference.

The catheter 28 is advanced with its distal end beneath the distal-most limb section (relative to the catheter position) as shown in FIG. 5C. The implant 22 is deployed from catheter as the catheter is withdrawn, causing the implant 22 to self-expand to engage the limb sections L1, L2 as shown in FIG. 5D.

According to an alternative deployment method, the implant 22 is deployed prior to the cuff 24 in a manner similar to that described in connection with FIGS. 5C and 5D. According to this embodiment, the cuff 24 may be a wrap-around device that is placed around the limbs L1, L2 after the implant 22 is positioned inside the limbs. The cuff 24 can then be secured in a tubular shape using sutures or other fastening means including but not limited to adhesives, Velcro-type fasteners or other mechanical fasteners.

The implant 22 and cuff 24 may be provided as a system together with additional instruments, which may include but are not limited to catheter 28 and/or other instruments useful for resecting the bowel and implanting the implant 22 and cuff 24. The system may additionally be provided with instructions for use instructing the user to implant the implant and cuff using methods disclosed herein and/or their equivalents.

While certain embodiments have been described above, it should be understood that these embodiments are presented by way of example, and not limitation. It will be apparent to persons skilled in the relevant art that various changes in form and detail may be made therein without departing from the spirit and scope of the invention. This is especially true in light of technology and terms within the relevant art(s) that may be later developed. Moreover, various features of the disclosed embodiments may be combined with one other or with additional features to create additional embodiments falling within the scope of the present invention.

Any and all patents, patent applications and printed publications referred to above, including those relied upon for purposes of priority, are incorporated by reference.

We claim:

1. A system for improving bowel function, the system comprising:
an elongate tube having first and second ends and an intermediate second between the first and second ends;
first and second anchors; and
instructions for use instructing a user to implant the elongate tube at a target location in the bowel by forming a pair of incisions in the bowel, passing the elongate tube through the first and second incisions, positioning the first and section ends at spaced apart locations within the bowel such that the intermediate section is positioned external to the bowel, and anchoring the first and second ends within the bowel using the anchors.

2. The system according to claim 1, wherein the first and second anchors include expandable members on the first and second ends.

3. The system according to claim 2, wherein the anchors are self-expandable from a radially compressed position.

4. The system according to claim 3, wherein the system includes a sheath positioned over the anchors to retain the anchors in the radially-compressed position, and wherein the instructions for use instruct the users to remove the sheath to allow the anchors to expand within the bowel.

5. The system according to claim 2, wherein the anchors are formed of mesh or braid.

6. The system according to claim 1, wherein the system further includes a catheter, and wherein the instructions for use instruct the user to insert the catheter into a natural orifice, to advance the catheter to the target location, and to advance the elongate tube through the catheter.

7. A system for forming an anastomosis between first and second ends of a resected bowel, comprising:
a tubular sleeve;
a tubular cuff; and
instructions for use instructing a user to draw the first and second ends into contact with one another, to position the tubular cuff surrounding the first and second ends, and to position the sleeve within the first and second ends such that the first and second ends are disposed between the sleeve and the cuff.

8. The system of claim 7, wherein the sleeve is self-expandable from a radially compressed position, and wherein the system further includes a sheath for retaining the sleeve in the radially compressed position.

9. The system of claim 7, wherein the sleeve is formed of mesh or braid.

10. The system of claim 7, wherein the instructions for use instruct the user to introduce the sleeve through a natural orifice and into contact with the first and second ends.

11. The system of claim 7, wherein the instructions for use instruct the user to introduce the cuff through an incision in a body wall and to insert the first and second ends into the cuff.

12. A method for improving bowel function, the system comprising:
providing an elongate tube having first and second ends and an intermediate section between the first and second ends;
forming a pair of incisions through the wall of a bowel;
passing the elongate tube through the first and second incisions, and positioning the first and second ends of the elongate tube at spaced apart locations within the bowel such that an intermediate section of elongate tube is positioned external to the bowel; and
anchoring the first and second ends within the bowel.

13. The method according to claim 12, wherein the first and second ends include expandable anchors, and wherein anchoring the first and second ends includes expanding the anchors within the bowel.

14. The method according to claim 13, wherein the method includes positioning the elongate tube within a sheath with the anchors in a radially compressed position, and wherein anchoring the first and second ends includes removing the sheath from the anchors.

15. The method of 14, wherein removing the sheath from the anchors causes the anchors to self-expand into contact with the bowel.

16. The method of claim 12, wherein the bowel includes a diseased section, and wherein the method includes forming a first one of the incisions upstream of the diseased section and forming a second one of the incisions downstream of the diseased section, and wherein following the anchoring step bowel contents flow through the elongate tube without passing through the diseased section.

17. The method of claim 12, further including the step of inserting a catheter into a natural orifice to an intestine, advancing the catheter through the intestine towards a first one of the incisions, and advancing the elongate tube through the catheter.

18. The method of claim 17, further including the step of advancing a first end of the elongate tube out of the bowel through the first incision, and withdrawing the first end of the elongate tube into the bowel through the second incision.

19. A method for forming an anastomosis between first and second ends of a resected bowel, comprising:
providing a tubular sleeve and a tubular cuff;
drawing the first and second ends of the resected bowel into contact with one another,
positioning the tubular cuff surrounding the first and second ends; and
positioning the sleeve within the first and second ends such that the first and second ends are disposed between the sleeve and the cuff.

20. The method of claim 19, wherein the method includes introducing the sleeve through a natural orifice and into contact with the first and second ends.

21. The system of claim 19, wherein the method includes introducing the cuff into an abdominal cavity through an incision in a body wall and inserting the first and second ends into the cuff.
